(19) United States
(12) Patent Application Publication
(10) Pub. No.: US 2022/0184674 A1
(43) Pub. Date: Jun. 16, 2022

(54) WASTE MASK TREATMENT DEVICE USING STERILIZATION PART

(71) Applicant: Jun Young JOUNG, Cheongju-si, Chungcheongbuk-do (KR)

(72) Inventor: Jun Young JOUNG, Cheongju-si, Chungcheongbuk-do (KR)

(21) Appl. No.: 17/054,174

(22) PCT Filed: Nov. 4, 2020
(86) PCT No.: PCT/KR2020/015321

§ 371 Date: Nov. 10, 2020

(30) Foreign Application Priority Data
Jul. 16, 2020 (KR) 10-2020-0088057

(51) Int. Cl. B09B 3/35; A61L 2/14; A62B 18/02; B02C 18/14; B09B 3/70; B09B 3/50

(52) U.S. Cl. CPC B09B 3/35; A61L 2/14; A62B 18/02; A61L 2202/26; B09B 3/70; B09B 3/50; A61L 2202/11; B02C 18/142

(57) ABSTRACT

The present invention relate to a waste mask treatment device using a sterilizing material, and more particularly, to a waste mask treatment device including: a body with an inlet adapted to insert a waste mask thereinto; a pulverization part located inside the body to pulverize the waste mask into pieces of mask; a sterilization part for emitting a sterilizing material for sterilizing the pieces of mask; and a collection part for collecting the pieces of mask. Accordingly, the waste mask treatment device is capable of being conveniently movable, being compact in size, and eliminating more than 99.99% of harmful bacteria living on the disposable mask through the sterilizing material.

### TECHNICAL FIELD

The present invention relates to a waste mask treatment device using a sterilizing material.

### BACKGROUND ART

Generally, medical waste is waste that is generated in medical facilities, while containing infectious materials harmful to the human bodies. In some cases, the medical waste is called infectious waste.

For example, the medical waste includes absorbent cotton, bandage, gauze, disposable syringe, infusion set, and so on, and they are already contaminated with blood, body fluids, excrements, and fluids.

Further, the medical waste may contain components harmful to the human bodies or cause infection, so that it is sorted from general waste and is thus treated under strict management. The most common method for treating the medical waste is an incineration method.

Even until now, the incineration method is the most popular method, and in Korea, most of medical waste is treated through incineration. Generally, an incineration device includes an incinerator for burning medical waste, a dust collector for purifying the exhaust gas generated during the burning, and a heat exchanger for cooling the exhaust gas.

A conventional continuous type medical waste treatment device is disclosed in Korean Patent No. 10-1117069, which includes: a pulverizing part for pulverizing medical waste; a heat treatment part for sterilizing the pulverized medical waste with high temperature steam pressure; an input and output part for supplying and discharging the pulverized medical waste, while keeping the steam pressure of the heat treatment part; and an odor removing part for removing the odor generated from the heat treatment part and having a burner for burning the odor emitted through the heat treatment part and a fan for introducing the odor into the burner.

In the conventional medical waste treatment device, however, germs may be distributed to the outside in the process of pulverizing the medical waste, and further, interiors of needles or tubes may be not perfectly sterilized.

Accordingly, there is a need to solve such problems. Further, conventional medical waste treatment methods are disclosed in Korean Patent No. 10-1926259 (issued on Nov. 30, 2018) entitled “medical waste treatment method using microwave and hot air” and Korean Patent No. 10-1779296 (issued on Sep. 11, 2017) entitled “screw type medical waste treatment method using microwave”.

The conventional medical waste treatment device is complicated in configuration and bulky in size, so that it is hard in using the device for household and in moving the device freely. Until now, on the other hand, there is no device for treating a used disposable mask.

### DISCLOSURE

### Technical Problem

Accordingly, the present invention has been made to solve the above-mentioned problems, and it is an object of the present invention to provide a waste mask treatment device that is capable of being conveniently movable, being compact in size, easily pulverizing a waste mask, and eliminating more than 99.99% of harmful bacteria living on the pieces of mask pulverized through circulation of a sterilizing material and irradiation of ultraviolet rays.

### Technical Solution

To accomplish the above-mentioned objects, according to the present invention, there is provided a waste mask treatment device including: a body with an inlet adapted to insert a waste mask thereinto; a pulverization part located inside the body to pulverize the waste mask into pieces of mask; a sterilization part for emitting a sterilizing material to allow the pieces of mask to be sterilized; and a collection part for collecting the pieces of mask.

According to the present invention, desirably, the waste mask treatment device further includes a transfer part for transferring the pieces of mask pulverized from the pulverization part so that the pieces of mask are exposed to the sterilizing material for a given set time.

According to the present invention, desirably, the waste mask treatment device further includes a circulation part located inside the body to circulate the sterilizing material.

According to the present invention, desirably, the collection part includes injectors located on an internal bottom of the body to emit the sterilizing material introduced thereinto to an interior of the collection part, so that if the pieces of mask are accumulated thereon, the sterilizing material permeates into the pieces of mask through the injectors and sterilizes the pieces of mask.

According to the present invention, desirably, the waste mask treatment device further includes a disinfection part located inside the body to supply at least any of a disinfectant sprayed or disinfection towel to the outside of the body.

According to the present invention, desirably, the waste mask treatment device further includes ultraviolet sterilization parts for sterilizing the pieces of mask accumulated on the collection part through ultraviolet rays.

### Advantageous Effects

According to the present invention, the waste mask treatment device can pulverize the waste mask into the pieces of mask with given sizes and allow the pieces of mask to be exposed to the sterilizing material, so that the pieces of mask can be evenly sterilized.

In addition, the waste mask treatment device can permeate the sterilizing material into the pieces of mask even if the pieces of mask are accumulated on the collection part, so that the pieces of mask accumulated on the collection part can be continuously sterilized.

Further, the waste mask treatment device can circulate the sterilizing material therein, so that the pieces of mask as well as the interior of the device can be sterilized.

Furthermore, the waste mask treatment device can irradiate the ultraviolet rays onto the pieces of mask if the pieces of mask are accumulated on the collection part, so that the pieces of mask can be continuously sterilized.

Moreover, the waste mask treatment device can disinfect the outer surfaces thereof as well as the user's hands through the disinfectant or disinfection towel after the waste mask has been treated, so that the device or the user's body can be prevented from being contaminated or infected.

Also, the waste mask treatment device can transfer the pieces of mask by a given distance, so that the pieces of mask can be evenly sterilized.

In addition, the waste mask treatment device can detect an amount of pieces of mask accumulated on the collection part, so that the pieces of mask can be prevented from being excessively accumulated on the collection part.

Further, the waste mask treatment device can stop operating the pulverization part through the emergency stop button if the foreign matters like clothes or gloves are caught to the pulverization part.

Furthermore, the waste mask treatment device can operate the pulverization part in a reverse direction through the discharge button if the foreign matters are caught to the pulverization part to fail to operate the pulverization part, so that the foreign matters can be discharged to the outside.

In addition, the waste mask treatment device can recognize the current state thereof through the touchscreen and allow the sterilization part, the transfer part, the pulverization part, the circulation part, the ultraviolet sterilization parts, and the disinfection part to be easily controlled through the controller.

### DESCRIPTION OF DRAWINGS

FIG. 1 is a schematic perspective view showing a waste mask treatment device according to the present invention.

FIG. 2 is a schematic perspective view showing a pulverization part, a transfer part, and a collection part in the waste mask treatment device according to the present invention.

FIG. 3 is a side sectional view showing the waste mask treatment device according to the present invention.

FIG. 4 is a side sectional view showing an operating state of the waste mask treatment device according to the present invention.

FIG. 5 is a side sectional view showing another example of the transfer part of the waste mask treatment device according to the present invention.

[Explanations on Reference Numerals]

| | |
|---|---|
| 1: body | 11: housing |
| 111: inlet | 112: internal space |
| 113: passage | 12: side wall |
| 14: partition wall | 15: internal bottom |
| 151: first through hole | 16: door |
| 161: locking member | 2: pulverization part |
| 21: pulverizing motor | 22: pulverizing cutter |
| 23: gear | 3,3′: transfer part |
| 31: driving roller | 32: conveyor belt |
| 35: transfer motor | 37: transfer panel |
| 4: collection part | 41: second through hole |
| 42: injectors | 5: sterilization part |
| 6: circulation part | 61: first blowing fan |
| 62: second blowing fan | 7: disinfection part |
| 71: disinfectant sprayer | 72: disinfection towel |
| 8: controller | 81: touchscreen |
| 82: emergency stop button | 83: forward rotation control button |
| 84: reverse rotation control button | S1: first detector |
| S2: second detector | S3: third detector |
| 9: ultraviolet sterilization part | P: pieces of mask |
| M: mask | |

### MODE FOR INVENTION

Hereinafter, the present invention will now be described in detail with reference to the attached drawings, and if it is determined that the detailed explanation on the well known technology related to the present invention makes the scope of the present invention not clear, the explanation will be avoided for the brevity of the description.

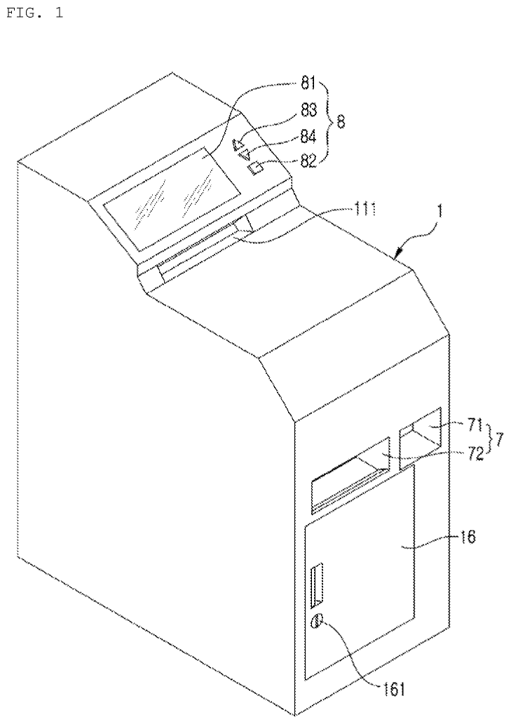

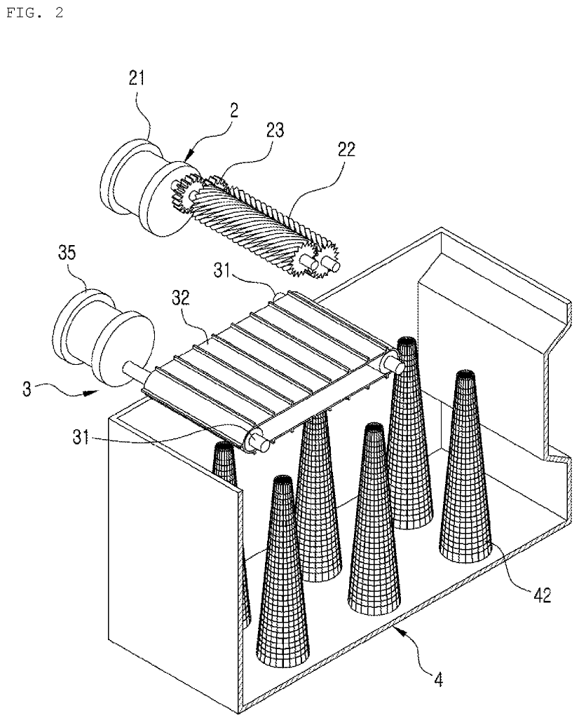

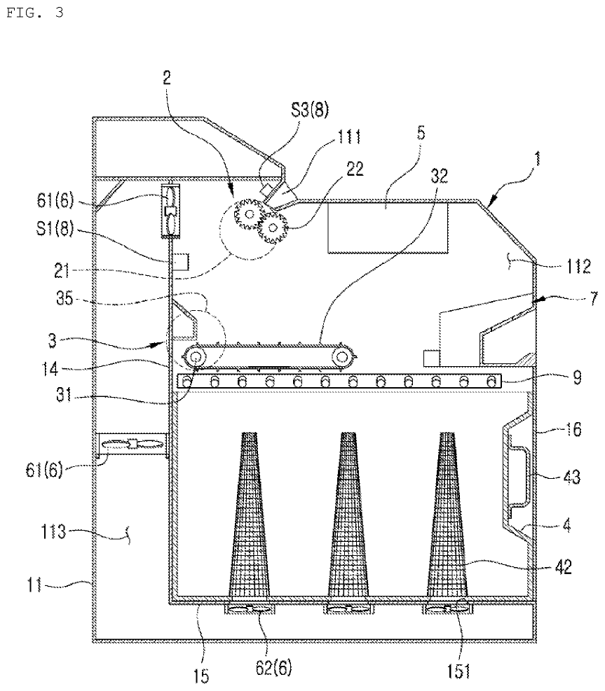

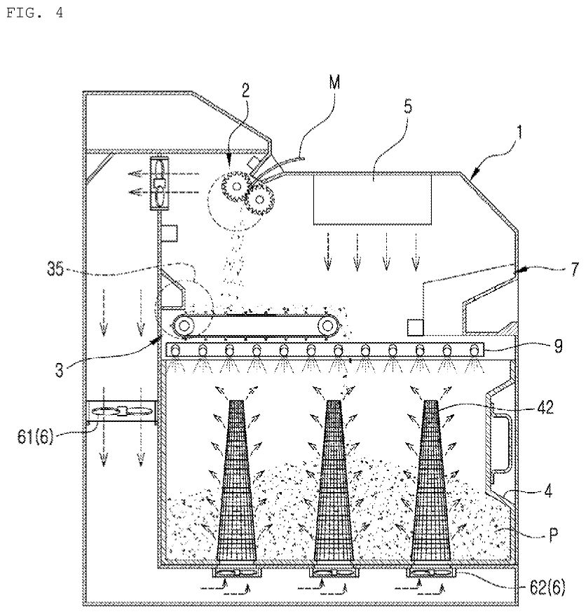

As shown in FIGS. 1 to 5, a waste mask treatment device according to the present invention includes a body 1, a pulverization part 2, a transfer part 3 or 3′, a collection part 4, a sterilization part 5, a circulation part 6, a disinfection part 7, ultraviolet sterilization parts 9, and a controller 8.

The body 1 constitutes an outer shape of the waste mask treatment device and includes a housing 11, a side wall 12, a partition wall 14, an internal bottom 15, and a door 16 in such a manner as to accommodate the pulverization part 2, the transfer part 3, the collection part 4, the sterilization part 5, the circulation part 6, the disinfection part 7, and the controller 8 thereinto.

The housing 11 constitutes an outer shape of the body 1 and includes an inlet 111 formed on top thereof to insert a waste mask (disposable mask) M thereinto and internal space 112 formed at the inside thereof to accommodate the pulverization part 2, the transfer part 3, the collection part 4, the sterilization part 5, the circulation part 6, the disinfection part 7, and the controller 8 thereinto.

The side wall 12 is a panel for partitioning the internal space 112 in a vertical direction, which is located closer to the right side of the housing 11 than the left side thereof. Accordingly, a pulverizing motor 21 and a transfer motor 35 as will be discussed later are located on the right space of the internal space 112 with respect to the side wall 12, and other components are on the left space of the internal space 112, so that pieces of masks P can be prevented from being caught to the motors, thereby avoiding malfunctions of the motors.

The partition wall 14 is located on the rear side of the internal space 112 in such a manner as to allow one end to come into close contact with the inner top surface of the housing 11 and to allow the other end to be connected to the internal bottom 15 to provide a passage 113 on the rear side of the inside of the housing 11. An explanation on the passage 113 will be given below.

The internal bottom 15 is spaced apart from bottom of the housing 11 by a given distance in such a manner as to allow one end to come into close contact with the inner front surface of the housing 11 and to allow the other end to be connected to the other end of the partition wall 14 to provide the passage 113 on the bottom side of the inside of the housing 11. Accordingly, the “L”-shaped passage 113 is extended from the rear side of the housing 11 to the bottom side thereof, and along the passage 113, a sterilizing material can move.

The door 16 opens and closes a given front area of the housing 11 and includes a locking member 161. Accordingly, the front surface of the housing 111 can be open and closed by means of the door 16, and the door 16 is locked by means of the locking member 161, so that the collection part 5 can be prevented from escaping from the housing 11 to the outside.

The pulverization part 2 is located in the internal space 112 of the housing 11 to pulverize the waste mask M into the pieces of mask P with a given size under control of the controller 8 and includes the pulverizing motor 21, pulverizing cutters 22, and gears 23.

The pulverizing motor 21 is located at the right side of the internal space 112 of the housing 11 in such a manner as to be connected to one of the gears 23 to rotate the pulverizing cutters 22 and is controlled in rotating direction and speed by means of the controller 8.

One pair of pulverizing cutters 22 are located at the left side of the internal space 112 of the housing 11 and are connected to the helical gears 23 in such a manner as to allow cutter blades thereof to engage with each other to pulverize the waste mask M into the pieces of masks P with the given size.

The gears 23 are located at the right side of the internal space 112 of the housing 11 to rotate the pulverizing cutters 22, and the gears 23 are connected to the pulverizing cutters 22 on one end of the pulverizing cutters 22 in such a manner as to engage with each other. One of the gears 23 is connected to the pulverizing motor 21. Accordingly, if the pulverizing motor 21 operates, the gears 23 rotate, and if the gears 23 rotate, the pulverizing cutters 22 rotate. Accordingly, if the pulverizing cutters 22 rotate forward, the waste mask M is caught between the pulverizing cutters 22 and is thus pulverized, and in this case, if the pulverizing cutters 22 rotate reversely, the waste mask M is discharged from the inlet 111. In detail, if foreign matters except the waste mask M enter the pulverizing cutters 22, the pulverizing cutters 22 rotate reversely to remove the foreign matters therefrom, and next, the waste mask M is pulverized again, thereby preventing the pulverizing cutters 22 from being damaged.

The transfer part 3 serves to transfer the pieces of mask P pulverized to a given speed and includes one pair of driving rollers 31, a conveyor belt 32, and the transfer motor 35.

One pair of driving rollers 31 are located at front and rear sides of the transfer part 3 on the left side of the internal space 112 of the housing 11 so as to drive the conveyor belt 32, and one of the driving rollers 31 is connected to the transfer motor 35. In this case, the driving roller 31 located at the front side of the transfer part 3 is located at the center of the internal space 112 to allow the pieces of mask P pulverized to fall down toward the center of the collection part 4.

The conveyor belt 32 is mounted on the driving rollers 31 to allow the pieces of mask P falling from the pulverizing cutters 22 to fall down toward the collection part 4, and further, the conveyor belt 32 has concave and convex portions formed on the outer surface thereof to easily transfer the pieces of mask P thereon. An explanation on the configuration of the conveyor belt 32 will be given later.

The driving motor 35 is located on the right side of the internal space 112 of the housing 11 to rotate the conveyor belt 32 and is connected to the driving roller 31 located at the rear side of the transfer part 3. A rotating speed of the driving motor 35 is controlled by means of the controller 8. An explanation on the control of the rotating speed of the conveyor belt 32 through the controller 8 will be given later.

If the transfer motor 35 operates, accordingly, the driving rollers 31 rotate, and if the driving rollers 31 rotate, the conveyor belt 32 operates. In this case, the rotating speed of the conveyor belt 32 is changed according to the rotating speed of the transfer motor 35, and accordingly, the moving speed of the pieces of mask M can be determined. For example, if the number of waste masks is small, the rotating speed of the transfer motor 35 becomes low, thereby decreasing the moving speed of the pieces of mask P, and contrarily, if the number of waste masks is large, the rotating speed of the transfer motor 35 becomes high, thereby increasing the moving speed of the pieces of mask P. As a result, the pieces of mask P can be prevented from being accumulated on the conveyor belt 32, thereby avoiding an interference with the pulverizing cutters 22.

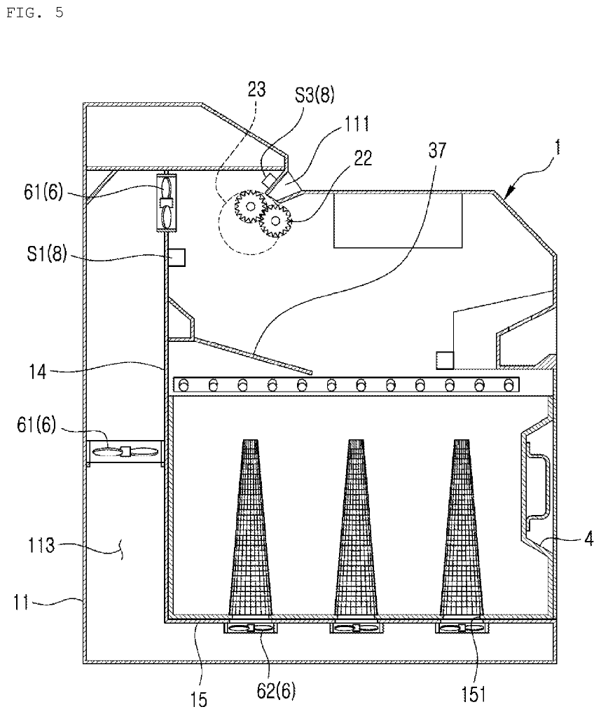

On the other hand, as shown in FIG. 5, another transfer part 3′ only with a plurality of transfer panels 37 is provided. In detail, the transfer panels 37 are short in length and have front ends inclined downward so that the pieces of mask P can fall down toward the center of the collection part 4. If the pieces of mask P fall down from the pulverization part 2, accordingly, they freely fall down along the transfer panels 37. In this case, the moving speed of the pieces of mask P can be determined according to the angles of the transfer panels 37, and accordingly, the transfer panels 37 are located with angles where the pieces of mask P can move to a relatively slow speed. As a result, the pieces of mask P can be exposed sufficiently to the sterilizing material before accumulated on the collection part 4, so that they can be easily sterilized. In this case, a first detector S1 as will be discussed later may not exist.

The collection part 4 is a container for collecting the pieces of mask P pulverized and has the shape of a generally rectangular parallelepiped that is open on top thereof and is concave backward from a given area of the front surface thereof in such a manner as to prevent a handle as will be discussed later from protruding therefrom. The collection part 4 is laid on the internal bottom of the body 1 and includes injectors 42 and a plurality of second through holes 41 connected to a plurality of first through holes 151 formed on the internal bottom of the body 1. Accordingly, the pieces of mask P falling from the transfer part 3 are collected to the collection part 4, and the sterilizing material is introduced into the collection part 4 through the first through holes 151 and the second through holes 41. An explanation on the collection and sterilization of the pieces of mask P will be in detail given later.

Also, the collection part 4 includes the injectors 42 and the handle.

The injectors 42 serve to supply and distribute the sterilizing material to an interior of the collection part 4.

Further, each injector 42 has the shape of a general cone, while having fine punched holes formed on an outer peripheral surface thereof or a structure with meshes, through which only the sterilizing material, not the pieces of mask P can pass. The injectors 42 are spaced apart from one another inside the collection part 4 at given intervals in such a manner as to allow the lower end peripheries thereof to be connected to the second through holes 41. Accordingly, the sterilizing material introduced from the first through holes 151 and the second through holes 41 enters the interiors of the injectors 42 and is then discharged from the injectors 42 to the outside, so that the pieces of mask P accumulated on the collection part 4 can be continuously sterilized.

The handle is located on the front surface of the collection part 4 to easily insert or draw the collection part 4 into or from the housing 11. Accordingly, the door 16 is open to easily insert or draw the collection part 4 into or from the housing 11 through the handle.

The sterilization part 5 is located inside the housing 11 above the transfer part 3 or 3′ to emit the sterilizing material therefrom and is controlled by means of the controller 8. If the sterilizing part 5 operates, accordingly, the sterilizing material is emitted to the inside of the body 1.

In this case, the sterilizing part 5 makes use of ozone, plasma ions, and ion clusters emitted by high voltage. If the sterilizing part 5 makes use of ozone, the ozone can be generated by means of silent discharge, electrolysis, photochemical method, high frequency electric field method, irradiation, and so on, but desirably, the silent discharge is used in consideration of energy efficiency, stability in performance, and conveniences in control. Further, the ion clusters are produced by ionizing oxygen molecules in the air, coupling the generated ions with water, and combining the oxygen ions with radicals. In detail, as negative/positive oxygen ions, hydroxyl radicals, and superoxide radicals have strong affinity, they are collected to shapes of groups to produce the ion clusters, and after that, they survive for about 120 seconds. While they are surviving for about 120 seconds, they dissolve and choke odorous components, bacteria, and fungi, and after that, they are reduced to oxygen or water molecules.

Accordingly, at least any one of the ozone, plasma ions, and ion clusters is circulated inside the body 1 to evenly sterilize the pieces of mask P.

The circulation part 6 is controlled by means of the controller 8 to circulate the sterilizing material in the interior of the body 1 and includes first blowing fans 61 and second flowing fans 62.

The first blowing fans 61 are located on an upper portion of the partition wall 14 under the sterilization unit 5 to circulate the sterilizing material in the interior of the body 1, and the rotating speeds of the first blowing fans 61 are controlled by means of the controller 8. An explanation on the control of the rotating speeds of the first blowing fans 61 will be in detail given later.

The second blowing fans 62 are located at the respective first through holes 151 on the underside of the internal bottom 15 to supply the sterilizing material to the interior of the collection part 4, and the rotating speeds of the second blowing fans 62 are controlled by means of the controller 8. If the first blowing fans 71 operate, accordingly, the sterilizing material moves downward along the passage 113 and then enters the injectors 42 through the second blowing fans 72, and next, the sterilizing material is emitted from the injectors 42 to the outsides. The sterilizing material emitted to the outsides of the injectors 42 moves toward the upper portion of the internal space 112 by means of the first blowing fan 61 located on the upper portion of the internal space 112 and then moves to the passage 113 again. If the sterilizing material moves to the upper portion of the internal space 112, in this case, it can sterilize the pieces of mask P being transferred. Further, the pulverizing cutters 22 are exposed to the sterilizing material circulated, so that they can be sterilized, together with the pieces of mask P.

The disinfection part 7 serves to disinfect the waste mask treatment device or the user's hand after the waste mask M has been sterilized and includes a disinfectant sprayer 71 and a disinfecting towel discharger 72.

The disinfectant sprayer 71 has a disinfectant (not shown) located inside the body 1 and an entrance formed on the front surface of the body 1 above the door 16, and an amount of disinfectant sprayed is controlled by means of the controller 8. After the user's hand or a towel is put into the disinfectant sprayer 71, in this case, if the disinfectant sprayer 71 operates through a touchscreen 81, the disinfectant is sprayed from the disinfectant sprayer 71 to disinfect the user's hand with which the waste mask M is put into the waste mask treatment device.

The disinfecting towel discharger 72 serves to discharge a towel with the disinfectant and has an entrance formed next to the disinfectant sprayer 71. The number of towels discharged is controlled by means of the controller 8. If the towel with the disinfectant is discharged from the disinfecting towel discharger 72, accordingly, the outer surfaces of the body 1 can be cleaned and disinfected, and if an amount of disinfectant applied to the towel is not sufficient, the towel may be put into the disinfectant sprayer 71 to allow the disinfectant to be sprayed thereonto.

The ultraviolet sterilization parts 9 serve to sterilize the pieces of mask P accumulated on the collection part 4 through ultraviolet rays, and they are located on both inner surfaces of the housing 11 between the conveyor belt 32 and the collection part 4 in such a manner as to be inclined downward toward the collection part 4. Accordingly, the pieces of mask M accumulated on the collection part 4 can be secondarily sterilized, thereby eliminating more than 99.99% of harmful bacteria living on the pieces of mask P.

The controller 8 is located inside the body 1 to control at least any one of the pulverization part 2, the transfer part 3, the sterilization part 5, the circulation part 6, the ultraviolet sterilization parts 9, and the disinfection part 7 and includes the touchscreen 81, an emergency stop button 82, a forward rotation button 83, a reverse rotation button 84, the first detector S1, and a second detector S2.

The touchscreen 81 is located with a given inclination on top of the body 1 behind the inlet 111. Further, the touchscreen 81 is adapted to input commands for controlling at least any one of the pulverization part 2, the transfer part 3, the sterilization part 5, the circulation part 6, and the disinfection part 7 thereto and is controlled by means of the controller 8. Through the touchscreen 81, accordingly, the rotating directions of the pulverizing cutters 22, the pulverizing speed, the transfer speed, the amount of sterilizing material emitted, the circulation speed of the sterilizing material, the emission time of the ultraviolet rays, and the operation of the disinfection part 7 can be controlled.

The emergency stop button 82 is located close to the touchscreen 81 on top of the body 1 to stop operating the pulverization part 2. If the foreign matters like clothes or gloves are caught to the pulverizing cutters 22, together with the waste mask M, accordingly, the emergency stop button 82 is pressed to stop operating the pulverization part 2.

The reverse rotation button 84 is located close to the touchscreen 81 on top of the body 1 to allow the rotating direction of the pulverizing cutters 22 to become reverse. If the reverse rotation button 84 is pressed in the state where the emergency stop button 82 is pressed to thus stop operating the pulverization part 2, accordingly, the pulverizing cutters 22 rotate reversely to discharge the foreign matters and the waste mask M caught thereto from the inlet 111.

The forward rotation button 83 is located close to the touchscreen 81 on top of the body 1 to allow the pulverizing cutters 22 to have a forward rotation direction. Even if the reverse rotation button 84 is pressed in the state where the foreign matters are caught to the pulverizing cutters 22, the foreign matters may not be discharged from the inlet 111, and in this case, the forward rotation button 83 and the reverse rotation button 84 are pressed sequentially to remove the foreign matters caught to the pulverizing cutters 22 little by little. Accordingly, the pulverizing cutters 22 can be prevented from being damaged.

The first detector S1 is located on the partition wall 14 under the first blowing fan 61 located on the upper portion of the body 1 to detect a height of the pieces of mask P accumulated on the conveyor belt 32 and to thus produce information on the height and is connected to the controller 8. Accordingly, the controller 8 receives the produced information from the first detector S1 and notifies the accumulated state of the pieces of mask P through the touchscreen 81. Further, the controller 8 controls the operation and transfer speed of the transfer part 3 on the basis of the produced information to prevent the pieces of mask P from being excessively accumulated on the conveyor belt 32.

The second detector S2 is located on the rear surface of the disinfection part 7 to detect a height of the pieces of mask P accumulated on the collection part 4 and to thus produce information on the height and is connected to the controller 8. Accordingly, the controller 8 receives the produced information from the second detector S2 and outputs collection time of the pieces of mask P accumulated on the collection part 4 through the touchscreen 81, thereby preventing the pieces of mask P from being excessively accumulated on the collection part 4.

In this case, the controller 8 further includes a third detector S3 for detecting the waste mask M inserted into the inlet 111. If the waste mask M is inserted into the inlet 111, accordingly, it is detected through the third detector S3, so that the pulverization part 2 automatically operates. If the waste mask M is not inserted into the inlet 111, in detail, the pulverization part 2 stops, and only when the waste mask M is inserted into the inlet 111, the pulverization part 2 operates. Accordingly, the life span of the pulverization part 2 can be extended, and also, energy can be saved.

Now, an explanation on operations of the waste mask treatment device according to the present invention will be given with reference to the attached drawings.

In a state where the collection part 4 is inserted into the body 1, the sterilization part 5 and the circulation part 6 operate through the touchscreen 81.

If a given amount of sterilizing material is filled in the body 1 through the operation of the sterilization part 5, the waste mask M is inserted into the inlet 111.

If the waste mask M is inserted into the inlet 111, it is detected by means of the third detector S3 to allow the pulverization part 2 to operate under the control of the controller 8.

If the pulverization part 2 operates, the waste mask M is caught to the pulverizing cutters 22 and is pulverized into the pieces of mask P with the given size.

The pieces of mask P pulverized are accumulated on the conveyor belt 32, and according to the amount of pieces of mask P accumulated, the transfer part 3 operates under the control of the controller 8.

If the transfer part 3 operates, the pieces of mask P moves forward at a given speed, and if they keep moving, they fall down into the collection part 4.

In this case, the sterilizing material serves to sterilize the pieces of mask P, while the pieces of mask P are moving. In detail, the transfer speed of the pieces of mask P is minimized to allow the pieces of mask P to be exposed to the sterilizing material as long as possible, so that the harmful bacteria remaining in the waste mask M can be sterilized to the maximum.

Even if the pieces of mask P are accumulated on the collection part 4, further, the sterilizing material permeates into the pieces of mask P through the injectors 42, thereby allowing the pieces of mask P to be continuously sterilized, and also, the pieces of mask P are sterilized once again through the ultraviolet rays emitted from the ultraviolet sterilization parts 9, thereby eliminating more than 99.99% of harmful bacteria.

As the sterilizing material is continuously circulated in the body 1, further, the interior of the body 1 can be prevented from being contaminated, and even if the collection part 44 and the pulverizing cutters 22 are not sterilized separately, they can be sterilized by means of the sterilizing material circulated in the body 1, so that they can be prevented from being contaminated.

In the description of the present invention, the corresponding parts in the embodiments of the present invention are indicated by corresponding reference numerals and the repeated explanation on the corresponding parts will be avoided.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A waste mask treatment device comprising:
a body with an inlet adapted to insert a waste mask thereinto;
a pulverization part located inside the body to pulverize the waste mask into pieces of mask;
a sterilization part for emitting a sterilizing material to allow the pieces of mask to be sterilized; and
a collection part for collecting the pieces of mask.

2. The waste mask treatment device according to claim 1, further comprising a transfer part for transferring the pieces of mask pulverized from the pulverization part so that the pieces of mask are exposed to the sterilizing material for a given set time.

3. The waste mask treatment device according to claim 1, further comprising a circulation part located inside the body to circulate the sterilizing material.

4. The waste mask treatment device according to claim 3, wherein the collection part comprises injectors located on an internal bottom of the body to emit the sterilizing material introduced thereinto to an interior of the collection part, so that if the pieces of mask are accumulated thereon, the sterilizing material permeates into the pieces of mask through the injectors and sterilizes the pieces of mask.

5. The waste mask treatment device according to claim 1, further comprising a disinfection part located inside the body to supply at least any of a disinfectant sprayed or disinfection towel to the outside of the body.

6. The waste mask treatment device according to claim 1, further comprising ultraviolet sterilization parts for sterilizing the pieces of mask accumulated on the collection part through ultraviolet rays.
